(19) United States
(12) Patent Application Publication
(10) Pub. No.: US 2023/0023197 A1
(43) Pub. Date: Jan. 26, 2023

(54) METHODS, SYSTEMS, AND APPARATUSES FOR THE DETECTION OF OXYGEN TOXICITY RELATED SYMPTOMS

(71) Applicant: UNIVERSITY OF CONNECTICUT, Farmington, CT (US)

(72) Inventors: KI H. CHON, Mansfield, CT (US); HUGO POSADA-QUINTERO, Vernon, CT (US)

(21) Appl. No.: 17/865,105

(22) Filed: Jul. 14, 2022

Related U.S. Application Data
(60) Provisional application No. 63/221,728, filed on Jul. 14, 2021.

(51) Int. Cl. A61B 5/00; G16H 50/20; A61B 5/0531

(52) U.S. Cl. CPC A61B 5/7267; G16H 50/20; A61B 5/0531; A61B 5/4266; A61B 5/721; A61B 5/7228; A61B 5/7282; A61B 5/742

(57) ABSTRACT

Methods, systems and apparatuses for determining oxygen toxicity in users is disclosed. Electrodermal activity (EDA) data from a sensor may be received. The EDA data may be indicative of one or more physiological signals derived from sweat gland activity of the user. Time-varying index values may be determined based on the EDA data. The time-varying index values may be compared to a threshold to determine if one or more of the values satisfies the threshold. Satisfying the threshold may indicate oxygen toxicity is occurring within the user. A notification may be caused to occur based on the time-varying index value satisfying the threshold. The notification may cause the user to take actions to reduce the potential for further oxygen toxicity.

### CROSS-REFERENCE TO RELATED APPLICATIONS

This application claims the benefit of U.S. Provisional Application No. 63/221,728, filed Jul. 14, 2021, which is incorporated herein by reference in its entirety.

### GOVERNMENT LICENSE RIGHTS

This invention was made with government support under Grant No. N00014-19-1-2209 awarded by the Office of Naval Research. The government has certain rights in the invention.

### BACKGROUND

Divers and submariners are generally required to ascend slowly from deep depths of water to mitigate the risk of nitrogen gas expanding at an uncontrollable rate. However, certain situations may dictate that the diver may need to ascend quickly, without completing any decompression stops, which may be time-consuming. Pre-breathing 100% oxygen is used as a way to ascend from deep depths of water while foregoing the completion of one or more decompression stops.

However, breathing 100% oxygen may cause oxygen toxicity in the diver or submariner. Oxygen toxicity is a condition resulting from breathing molecular oxygen (O₂) at increased partial pressures. Oxygen toxicity may lead to seizures, diaphoresis, numbness in joints, as well as other symptoms.

### SUMMARY

It is to be understood that both the following general description and the following detailed description are exemplary and explanatory only and are not restrictive. Methods, systems, and apparatuses for the detection of seizure and oxygen toxicity related symptoms using electrodermal activity sensors are described herein. The present disclosure relates to the early detection of seizures and/or other oxygen toxicity-related symptoms (e.g., for use by divers and submariners).

Disclosed is a method. The method may include receiving electrodermal activity (EDA) data of a user. The EDA data may be sent by a sensor. The EDA data may be received by a computing device. The method may include determining a time-varying index value. The time-varying index value may be based on the EDA data. The method may include determining that the time-varying index value satisfies a threshold. The method may include causing a notification to be displayed. The notification may be displayed based on the time-varying index value satisfying the threshold.

Additional advantages will be set forth, in part, in the description which follows or may be learned by practice. The advantages will be realized and attained by means of the elements and combinations particularly pointed out in the appended claims.

### BRIEF DESCRIPTION OF THE DRAWINGS

The accompanying drawings, which are incorporated in and constitute a part of this specification, together with the description, serve to explain the principles of the methods, systems, and apparatuses described herein:

FIG. 1 shows an example system;

FIG. 2 shows an example hardware design;

FIG. 3 shows an example signal processing procedure;

FIGS. 4A and 4B show an example graphical recording;

FIGS. 5A-5D show an example recording;

FIGS. 6A-6C show an example graphical recording;

FIGS. 7A and 7B show an example recording;

FIG. 8 shows an example graphical recording;

FIGS. 9A and 9B show an example graphical recording;

FIG. 10 shows an example user interface;

FIG. 11 shows a flowchart of an example method; and

FIG. 12 shows a flowchart of an example method.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. Similarly, when values are expressed as approximations, by use of the antecedent “about,” it will be understood that the particular value forms another configuration. It will be further understood that the endpoints of each of the ranges are significant both in relation to the other endpoint, and independently of the other endpoint.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal configuration. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented to achieve the methods described herein. Furthermore, a computer program product on a computer-readable storage medium (e.g., non-transitory) having processor-executable instructions (e.g., computer software) embodied in the storage medium may also be implemented to process any of the methods described herein. Any suitable computer-readable storage medium may be utilized including hard disks, CD-ROMs, optical storage devices, magnetic storage devices, memristors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the functions specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Blocks of the block diagrams and flowcharts support a device or combinations of devices for performing the specified functions, combinations of steps for performing the specified functions, and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

Provided herein are methods, systems, and apparatuses for the detection of seizure and/or oxygen toxicity related symptoms using electrodermal (EDA) activity data from users. EDA is increasingly used as a measure of sympathetic function due to the accuracy of the measurement. In consideration of this, it may be beneficial to identify methods, systems, and/or apparatuses for using EDA to identify symptoms related to oxygen toxicity. In addition, it may be beneficial to identify methods, systems, and/or apparatuses to provide a user (e.g., a diver, submariner or other person below the surface of a body of water) with a notification that an impending seizure or other symptoms may result based on oxygen toxicity, for example.

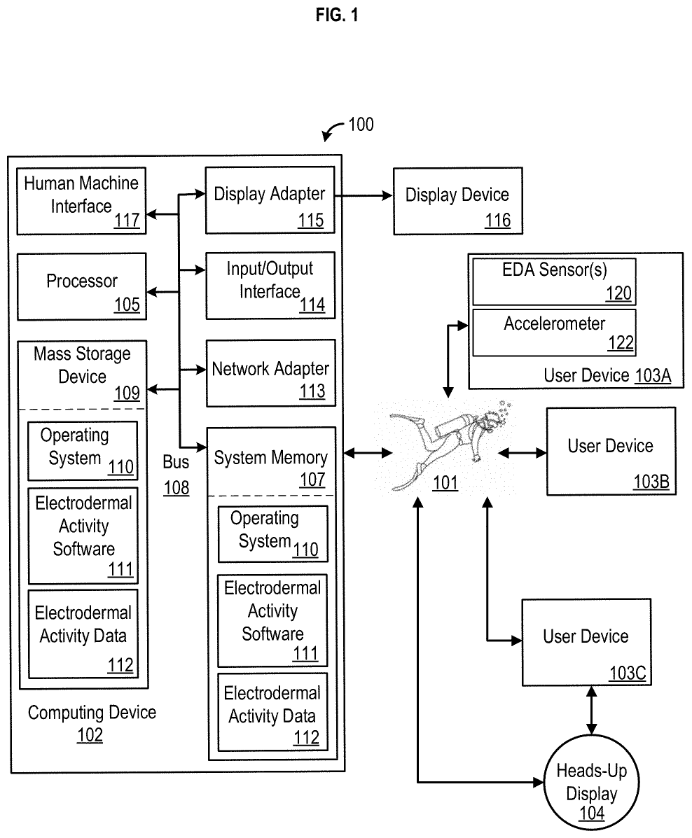

FIG. 1 shows an example system 100 for determining the potential for oxygen toxicity in a user (e.g., a user 101). For example, the example system 100 may be representative of a system for the collection and evaluation of EDA data of the user 101. The example system 100 may also be representative of a system for the collection and evaluation of other data (e.g., accelerometer data). The example system 100 may be a system for the prediction and/or detection of central nervous system oxygen toxicity (CNS-OT) in users (e.g., a diver or submariner). Although only certain devices and/or components are shown, the system 100 may comprise a variety of other devices and/or components that support a wide variety of functions, such as network and/or communication functions. Those skilled in the art will appreciate that the present systems and methods may be used in various types of networks and systems that employ both digital and analog equipment.

The system 100 may include one or more sensors 120, 122 affixed to a user 101. For example, a plurality of sensors 120, 122 may be affixed to the user 101. For example, the plurality of sensors may be affixed to the user 101 via one or more user devices 103A-C, such as user device 103A. In another example, one or more sensors of the plurality of sensors 120, 122 may be affixed to different objects (e.g., different user devices 103A-C) associated with the user 101. For example, the user device 103A may be a wearable device, such as a wrist-worn adornment (e.g., a wristband, a smartwatch, or a dive computer), a torso-worn adornment (e.g. a smart shirt or other device capable of positioning the sensors along a portion of the torso of the user 101), a facial-worn adornment, or the like. For example, each sensor of the plurality of sensors 120, 122 may be one or more of an EDA sensor 120, an accelerometer 122, or another form of sensor.

For example, each EDA sensor 120 may detect EDA data from the user 101. For example, each EDA sensor 120 may be configured to have all or at least a portion of the sensor positioned along the skin of the user 101. For example, each EDA sensor 120 may include one or more electrodes (not shown). For example, the electrodes may be stainless steel electrodes. In other examples, the electrodes may be made from another material. The electrodes may be communicably coupled to the EDA sensor 120 via one or more wires. For example, the electrodes may be placed on one or more of the finger(s), scapula, sternum, instep, and/or another portion of the skin of the body of the user 101. For example, the user device 103A may include two EDA sensors 120, however fewer or greater numbers of EDA sensors 120 on the user device 103A or on other user devices (e.g., user devices 103B and/or 103C) is contemplated within the scope of this disclosure. For example, the EDA data may comprise or be derived from one or more physiological signals of the user 101. For example, the one or more physiological signals may be derived from sweat gland activity detected by the EDA sensor 120 along the skin of the user 101.

The system 100 may also include an accelerometer 122 affixed to the user 101. The accelerometer 122 may be affixed to any object associated with the user 101. For example, the accelerometer 122 may be part of the user device 103A, another user device 103B-C, or another wearable device attached to the user 101. For example, the accelerometer 122 may detect accelerometer data associated with the user 101. For example, the accelerometer 122 (or the user device 103A or wearable device containing the accelerometer 122) may send accelerometer data. For example, the accelerometer data may be sent to a computing device 102 and/or one or more of the user devices 103A-C via a wireless or via wired internal or external transmission. The accelerometer data may be associated with a motion threshold, for example. As an example, the motion threshold may be indicative of a level of motion associated with the user 101 or a portion of the user 101. The motion threshold may be predetermined, for example. For example, the motion threshold may dynamically change based on dive patterns of the user 101. For example, the motion threshold may dynamically change by the use of machine learning techniques based on prior acceleration data of the user 101. The user device 103A, or another wearable device, may also include additional sensors, such as an electromyography sensor(s) and/or an electrocardiography sensor(s) that may each obtain associated data of the user 101.

The user device 103A may communicate with the computing device 102. The computing device 102 may be a dive computer, a mobile phone, a smartphone, a tablet computer, a smartwatch, a dive watch, or similar device. The user device 103A may communicate via wired or wireless communication with the computing device 102, For example, the user device 103A may wirelessly communicate with the computing device 102 via a wireless communication signal using a wireless communication protocol (e.g., Bluetooth®, Bluetooth Low Energy (BLE), radio frequency (RF) (e.g., electromagnetic RF), WiFi, or any other known wireless communication protocol). For example, the user device 103A may send or transmit the EDA sensor data, the accelerometer data, and any other sensor data from the user device 103A to the computing device 102.

The computing device 102 may collect and send the data from the sensors (e.g., sensors 120, 122 and/or other sensors) to one or more external devices, such as user devices 103B and 103C, for example. As another example, the user device 103A may communicate (via wire(s) or wirelessly) directly with a heads-up display 104 associated with the user device 103C. For example, the user device 103A may communicate with the heads-up display 104 via the user device 103C. It is understood that any of the user devices 103A and 103B may also be associated with a heads-up display. For example, the heads-up display 104 may be configured to be part of a dive mask or dive helmet that may be worn by the user 101. For example, the heads-up display 104 may include a notification feature. As an example, the notification feature may be text, one or more colors of lights, a sound, and/or a vibration. The notification between the user device 103A and the computing device 102 may aid in the detection of signal corruption, for example. As a further example, the notification may provide an index to warn the user 101 of a risk of developing CNS-OT symptoms.

The computing device 102 may comprise one or more processors 105 or processing units, a system memory 107, and a system bus 108 that couples various system components of the computing device 102, including the processor 105 to the system memory 107. In the case of multiple processors 105, the system may utilize parallel computing.

The system bus 108 represents one or more of several possible types of bus structures, including a memory bus or memory controller, a peripheral bus, an accelerated graphics port, and a processor or local bus using any of a variety of bus architectures. By way of example, such architectures may comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, a Peripheral Component Interconnects (PCI), a PCI-Express bus (USB) and the like.

The system bus 108 may also be implemented over a wired or wireless network connection to each of the subsystems, including the processor 105, a mass storage device 109, an operating system 110, EDA software 111, EDA data 112, a network adapter 113, an Input/Output (I/O) interface 114, a display adapter 115, a display device 116, and a human machine interface 117. It is understood that the system bus 108 and each of the aforementioned subsystems may be contained within each of the user devices 103A-C at physically separate locations, connected through buses of this form; in effect implementing a fully distributed system.

The computing device 102 may operate on and/or comprise a variety of computer-readable media (e.g., non-transitory computer-readable media). Computer-readable media may be any available media that is accessible by the computing device 102 and comprises both volatile and non-volatile media and removable and non-removable media. The system memory 107 may comprise computer-readable media in the form of volatile memory and removable and non-removable media. The system memory 107 may comprise computer-readable media in the form of volatile memory, such as random access memory (RAM), and/or non-volatile memory, such as read only memory (ROM). The system memory 107 typically contains data and/or program modules, such as an operating system 110 and EDA software 111 that are accessible to and/or are operated on by the one or more processors 105.

The EDA software 111 may be configured to receive the one or more physiological signals from the one or more sensors (e.g., the sensors 120, 122 and any other sensors associated with the user 101). The EDA software 111 may interpret the physiological signals into data (e.g., EDA data and/or time-varying index values of the EDA data) and may compare the data to one or more thresholds to determine if the threshold has been satisfied (e.g., the data is greater than or greater than or equal to the threshold or less than or less than or equal to the threshold, depending on the particular data and the threshold it is being compared to). For example, the data may be time-varying index values determined by the EDA software 111 based on the EDA data and the threshold may be associated with index values that may correspond to oxygen toxicity of the user 101. Accordingly, the oxygen toxicity of the user 101 may be determined by the EDA sensor software 111.

The system memory 107 may also contain accelerometer software (not shown). For example, the accelerometer software may remove motion artifacts from the physiological signals/data. It is understood that motion artifacts may be associated with a voluntary and/or involuntary motion of the user 101. The motion artifacts in the physiological signals may cause erroneous EDA data and correspondingly erroneous time-varying index values that, if compared to the threshold value(s) may incorrectly indicate oxygen toxicity of the user 101. The removal of motion artifacts may be beneficial to mitigate the risk against any false readings that may result in an incorrect notification indicating oxygen toxicity in the user 101 and a risk of developing any CNS-OT related symptoms by the user 101.

The computing device 102 may also comprise other removable/non-removable, volatile/non-volatile computer storage media. By way of example, the mass storage device 109 may provide non-volatile storage of computer code, computer readable (e.g., processor-executable) instructions, data structures, program modules, and other data for the user device 102. For example, the mass storage device 109 may be a hard disk, a removable magnetic disk, a removable optical disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 109, including by way of example, the operating system 110, EDA software 111, and EDA data 112. EDA data 112 may include the one or more physiological signals of the user 101 including historical EDA data for the user. The EDA data may also include time-varying index values for the user 101. The time-varying index values for the user 101 may also include historical time-varying index values for the user 101. The EDA data may also include accelerometer data for the user 101. The EDA data may also include one or more thresholds to compare the data (e.g., the EDA data, the accelerometer data, other data, etc.) to. The thresholds may be static or dynamic thresholds. For example, the dynamic thresholds may be determined based on historical data. For example, the time-varying index value thresholds may be dynamic thresholds determined based on historical time-varying index values for the user 101.

The display device 116 may also be connected to the system bus 108 via an interface, such as the display adapter 115. It is contemplated that the computing device 102 may have more than one display adapter 115 and the computing device 102 may have more than one display device 116. For example, a display device 116 may be a monitor, an LCD (Liquid Crystal Display), light emitting diode (LED) display, smart glass, or a projector. In addition to the display device 116, other output peripheral devices may comprise components, such as speakers (not shown) and a printer (not shown) which may be connected to the computing device 102 via the Input/Output interface 114. Any step and/or result of the methods may be output in any form to an output device. Such output may be any form of visual representation, including, but not limited to, textual, graphical, animation, audio, tactile, and the like. The display device 116 and the computing device 102 may be part of one device, or separate devices.

For purposes of illustration, application programs and other executable program components, such as the operating system 110 are illustrated herein as discrete blocks, although it is recognized that such programs and components reside at various times in different storage components of the computing device 102, and are executed by the data processor(s) of the computing device 102. An implementation of the EDA software 111 and/or the EDA data 112 may be stored on or transmitted across some form of computer-readable media. Any of the disclosed methods may be performed by computer-readable, processor-executable instructions embodied on computer-readable media. Further, any of the components or functions of the computing device 102 may equally be a part of and implemented in any one or each of the user devices 103A-C.

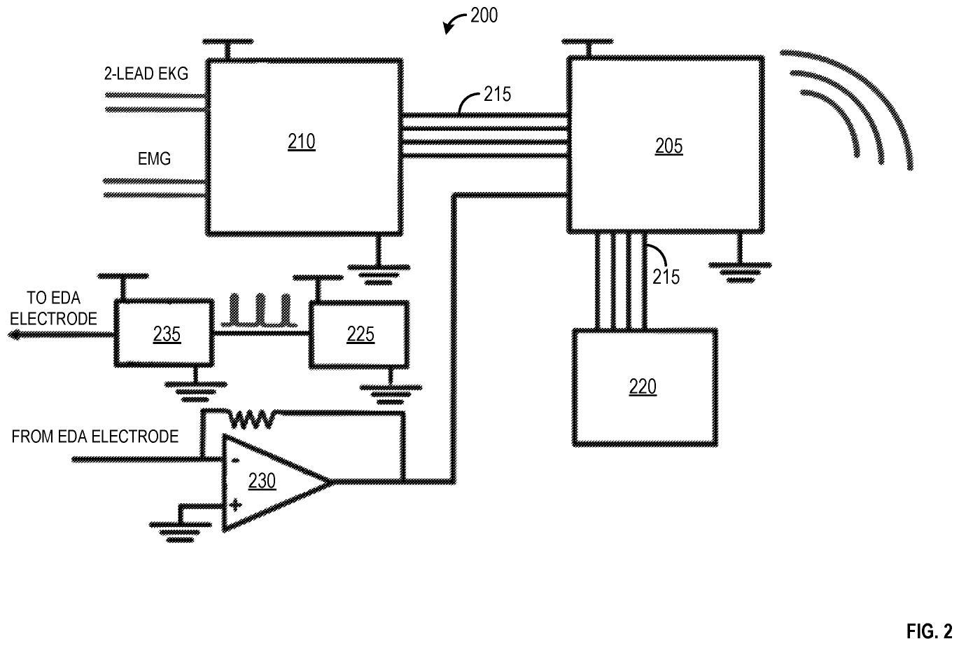

FIG. 2 shows an example circuit design 200 representative of at least a portion of the hardware design of the user device 103A. As an example, the portion of the hardware design of the user device 103A may be an EDA analog circuit. For example, each of the accelerometer 122 and the EDA sensor 120 may be associated with the user device 103A. For example, the accelerometer 122 and the EDA sensor 120 may be disposed within the user device 103A. The user device 103A may be configured to determine EDA data of the user 101 from the EDA sensor 120. Additionally, the user device 103A may include additional sensors (not shown) and may determine electrocardiogram and/or electromyographic signals of the user 101 as well. For example, the additional sensors may be disposed within the user device 103A. For example, the user device 103A may be a wearable device, such as a wrist-worn adornment (e.g., a wristband, bracelet, smartwatch, or dive computer), a torso-worn adornment (e.g. a smart shirt or other device capable of positioning the sensors along a portion of the torso of the user 101), a facial-worn adornment, or the like. For example, the user device 103A may be worn around a wrist of the user 101.

The user device 103A may communicate (via wire(s) or wirelessly) with the computing device 102. The user device 103A may also communicate (wired or wirelessly) with any of the other user devices 103B and 103C, for example. For example, the user device 103A may communicate wirelessly via a wireless communication signal using a wireless communication protocol (e.g., Bluetooth®, BLE, radio frequency (RF) (e.g., electromagnetic RF), WiFi, or any other known wireless communication protocol).

In certain examples, the user device 103A may include a microcontroller 205. The microcontroller 205 may communicate with an analog-to-digital converter chip 210 through a serial peripheral interface (SPI) 215. The microcontroller 205 may be a BGM121 microcontroller, for example. As another example, the microcontroller 205 may be an analog-to-digital converter (ADC) Bluetooth® microcontroller. The analog-to-digital converter chip 210 may be an ADS1298 ADC, for example. As a further example, the analog-to-digital converter chip 210 may be set to sample at 1 KHz. The analog-to-digital converter chip 210 may sample the output of an EDA analog circuit through an ADC so that captured information from both the EDA analog circuit and the analog-to-digital converter chip 210 may be sent to a flash memory 220. For example, the flash memory 220 may be representative of a NAND component. The captured information may be sent (via wire(s) or wirelessly) to the flash memory 220 via the SPI 215. The captured information may also be sent via wire(s) or wirelessly) to the computing device 102 and/or any of the user devices 103B and 103C, for example.

The user device 103A may further include a silicon oscillator 225 as a component of the circuit design 200. For example, the silicon oscillator 225 may be an LT6991 oscillator. The silicon oscillator 225 may be configured to produce a square wave with a frequency, duty cycle, and amplitude that may be changed. For example the frequency, duty cycle, and amplitude associated with the silicon oscillator 225 may be changed depending on external reference voltages and/or resistance values applied to one or more pins associated with a chip of the silicon oscillator 225.

For example, in the instance wherein the frequency of an output wave may be 100 Hz with a duty cycle of 50% and an amplitude of 1.5 V with a 0V offset associated with the circuit design 200, the output of the silicon oscillator 225 may be fed into a drain of a MOSFET (not shown). The MOSFET may be configured to shift a level of a signal so that oscillations never fall below a 0V threshold, for example. For example, the MOSFET may be a ZXM61M02FCT MOSFET.

The circuit design 200 of the user device 103A may further include a first op amp 230. The signal may be fed into a first op amp 230. The first op amp 230A may filter the square wave input into a sine wave, for example. Thus, an output from one of the EDA sensors 120 may be a sine wave. For example, a receiving electrode for one or more of the EDA sensors 120 may be fed to a second op amp (not shown). The second op amp may be configured as a transimpedance amplifier, for example. For example, each of the first op amp 230 and the second op amp may be an ADA4505 op amp.

The second op amp may convert a current going into a negative input terminal into a voltage that may be detected. As the amplitude of the current may be on the order of milliamps, the second op amp may be able to amplify the current by a significant amount, such as 200 times the original amount, so that a voltage may be more easily detected.

The circuit design 200 of the user device 103A may further include an envelope detector (not shown). The envelope detector may be added to the output of the second op amp since the current may oscillate similar to a sine wave and a change in amplitude over time is the measured value considered by the circuit design 200. The measured value considered by the circuit design 200 may be the TVSymp, for example. For example, the envelope detector may be configured to measure the positive peaks of the current and create a continuous positive output showing where changes in the amplitude may have occurred. The output may allow for the ADC of the microcontroller 205 to measure the EDA data signal, for example.

The circuit design 200 of the user device 103A may further include a level shifter (not shown) and low-pass filter (LPF) 235. The level shifter and LPF 235 may be configured to communicate with the silicon oscillator 225, as well as an EDA electrode (not shown). For example, the level shifter and LPF 235 may send signals to the EDA electrode. The first op amp 230 may also receive communication from the EDA electrode, for example. For example, the first op amp 230 may receive signals from the EDA electrode. As an example, the signals sent and/or received from the EDA electrode may be signals associated with EDA (e.g., EDA data). For example, the signals may be physiological signals of the user 101.

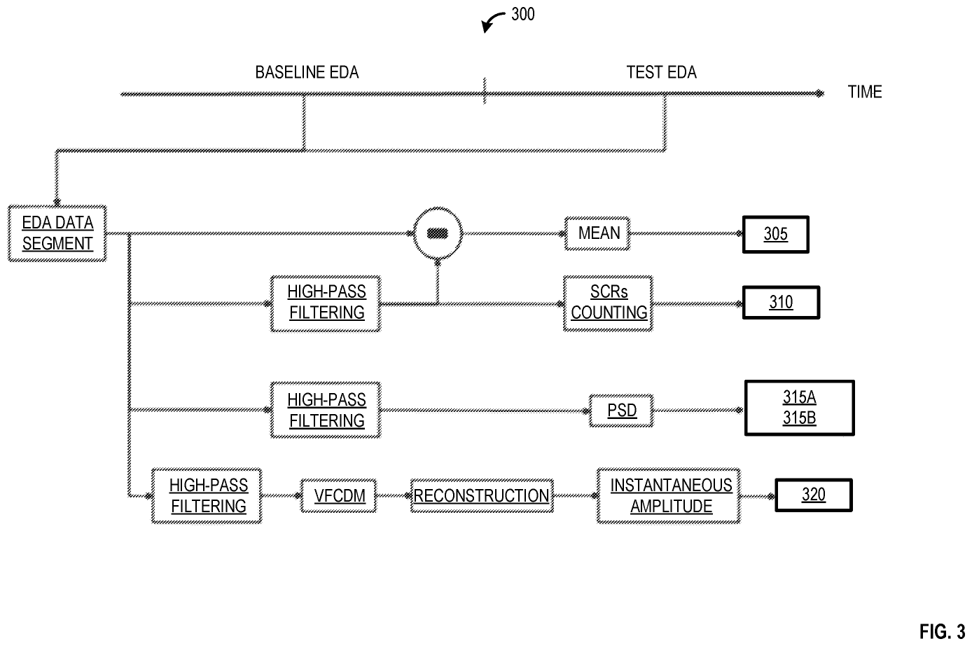

FIG. 3 shows an example signal processing procedure 300 to compute a variety of EDA indices. For example, the signal processing procedure 300 may be completed by the computing device 102, the user device 103A or any other device of the system 100. For example, a skin conductance level (SCL) 305 may be determined by calculating a mean of a plurality of collected EDA data segments received from the EDA sensor(s) 120. The plurality of EDA data segments may be collected by the EDA sensor(s) 120 during a non-stimulation rest period of the user 101, for example. For example, SCL may be a measure related to slow shifts associated with EDA data segments.

The signal processing procedure 300 may further be used to determine a number of skin conductance responses (NS.SCRs) 310. The NS.SCRs 310 may be determined by counting a number of skin conductance responses (SCRs) that may exist within a period of time. The SCRs may be associated with EDA data and may be indicative of physiological responses of the user 101 detected by the EDA sensor(s) 120. The counting of the number of SCRs may occur once a high-pass filtering process of the EDA data segment(s) has been completed, for example. The counting of the number of SCRs may occur post-stimuli, for example, and may be a tonic measurement. For example, SCRs may be rapid transient events contained in an EDA data signal. As another example, event-related SCRs (ER-SCRs) may be counted as well, wherein the ER-SCRs may occur immediately after the stimulus.

The signal processing procedure 300 may further be used to determine time-varying spectral analysis (TVSymp) 320 of EDA data (e.g., a time varying index value of the EDA data). For example, the TVSymp 320 may be determined upon completion of a high-pass filtering process of the EDA data segment(s), after which a power spectral density (PSD) may be determined. The determination of the PSD allows for either electrodermal activity-derived sympathetic (EDASymp) data 315A and/or EDASympn data 315B to be determined.

TVSymp 320, for example, may be determined by down-sampling and high-pass filtering EDA data segment(s) prior to a time-varying analysis so that any outlying trends may be removed from the data. A variable frequency complex demodulation (VFCDM) method may be implemented on the data so that a time-varying spectra of the EDA data may be obtained. The resulting time-varying spectral amplitudes obtained at each frequency interval may be analyzed so that a bandwidth most reflective of sympathetic dynamics may be determined. In certain examples, the spectral amplitudes may not be within fixed frequency bands. The spectral amplitudes may be representative of an evolution of a spectral amplitude that may change within a range of frequencies based on time. For example, the frequencies may depend on a sampling frequency (fs). As another example, fs may be equal to 2 HZ, which may be used to center the spectral frequencies on 0.04 Hz, 0.12 Hz, 0.2 Hz, 0.28 Hz, 0.36 Hz, 0.44 Hz, 0.52 Hz, 0.6 Hz, 0.68 Hz, 0.76 Hz, 0.84 Hz, and 0.92 Hz.

As an example, a modified TVSsymp (MTVSymp) may be determined by taking the derivative of the EDA data so that CNS-OT seizures may be better captured. The MTVSymp may be used as an index, for example. For example, features from a derivative of a phasic EDA (dphEDA) may be first computed as follows:

$$dphED A ( n ) = \frac{P ( n - 2 ) - 8 \cdot P ( n - 1 ) + 8 \cdot P ( n + 1 ) - P ( n + 2 )}{12 \cdot ( \frac{1}{fs} )}$$

where fₛ may be the sampling frequency (e.g., at 4 Hz), and P may be a phasic component of the EDA data (e.g., phEDA). For example, dphEDA(n) may be calculated by using a moving window of 1.25 seconds and by considering a sampling rate, as well as what may be considered to be a typical rise time of SCRs (e.g., 1-5 seconds). The index may be based on a spectral analysis of EDA and may be designed to remove pre-stimuli induced EDA data values, which may also be considered as baseline EDA data values, for example. Based on TVSymp, the following can be computed:

MTVSymp=max(0,a(t)−mean(a(t−w:t)))

where a is TVSymp and w is a time window. For example, five seconds may be the optimal value for w in the determination of MTVSymp. As another example, a mean MTVSymp value may be computed as an index of sympathetic reaction to stimuli.

The TVSymp index may demonstrate lower intra-subject variability compared to time-domain measures of EDA, for example. For example, the TVSymp may also demonstrate higher consistency and sensitivity to orthostatic and cognitive stress compared to SCL and NS.SCRs. Each of the TVSymp, EDASymp, EDASympn, SCL, and NS.SCRs may be utilized to evaluate a suitability of identified indices so that a sympathetic function may be assessed for types of sympathetic reactions that may be elicited by oxygen toxicity (e.g., CNS-OT), for example.

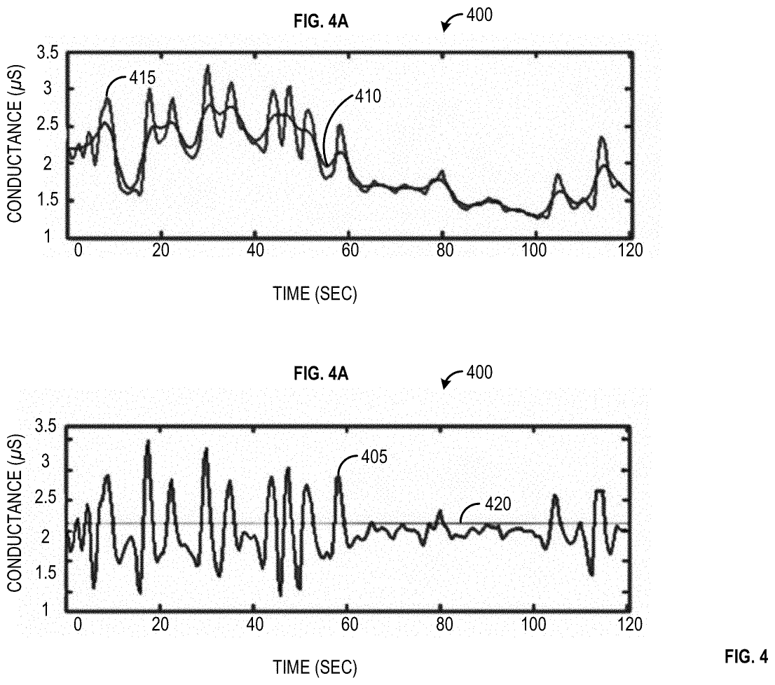

FIGS. 4A and 4B show an example graphical representation of collected EDA data. For example, the EDA data may be collected from the one or more EDA sensors 120 of FIG. 1. For example, FIGS. 4A and 4B may be demonstrative of a process for computing a time range (e.g., two minutes) of EDA data so that SCL and/NS.SCRs 405 may be determined. As an example, FIG. 4A shows measures of tonic EDA 410. The SCL may be determined based on the mean of tonic EDA 410. The tonic EDA 410 may be determined based on the raw EDA data 415, for example. The raw EDA data may be collected by the one or more EDA sensors 120 from the user 101. As another example, the NS.SCRs 405 may be determined based on removing the tonic EDA components from the raw EDA data. FIG. 4B shows a threshold 420 that may be used to determine which of the NS.SCRs 405 may be considered a positive response, for example.

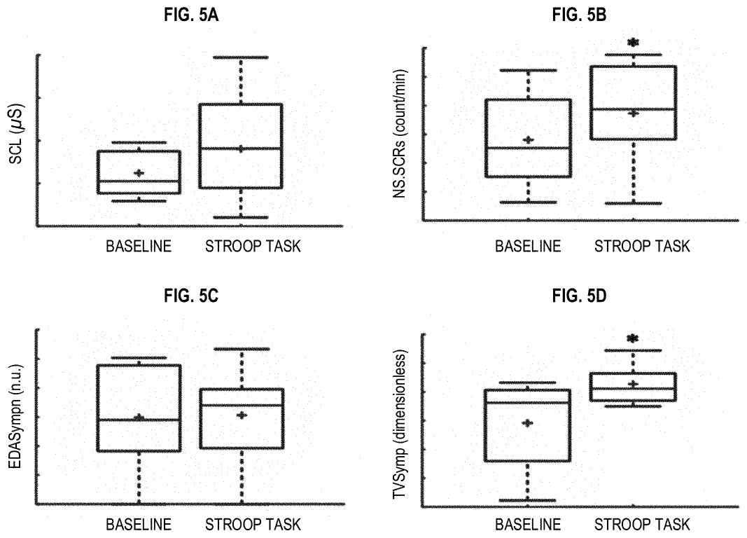

FIGS. 5A-5D show example box plots with obtained measurements for baseline values and Stroop task stages. For example, a Stroop task is a psychological experiment that may be used to make comparisons associated with EDA data. As an example, FIG. 5A shows a comparison associated with SCL values. FIG. 5B shows a comparison associated with NS.SCRs values. FIG. 5C shows a comparison associated with EDASympn values. FIG. 5D shows a comparison associated with TVSymp values. For each of the FIGS. 5A-5D it is understood that a (*) may denote a significant difference that may exist between stages.

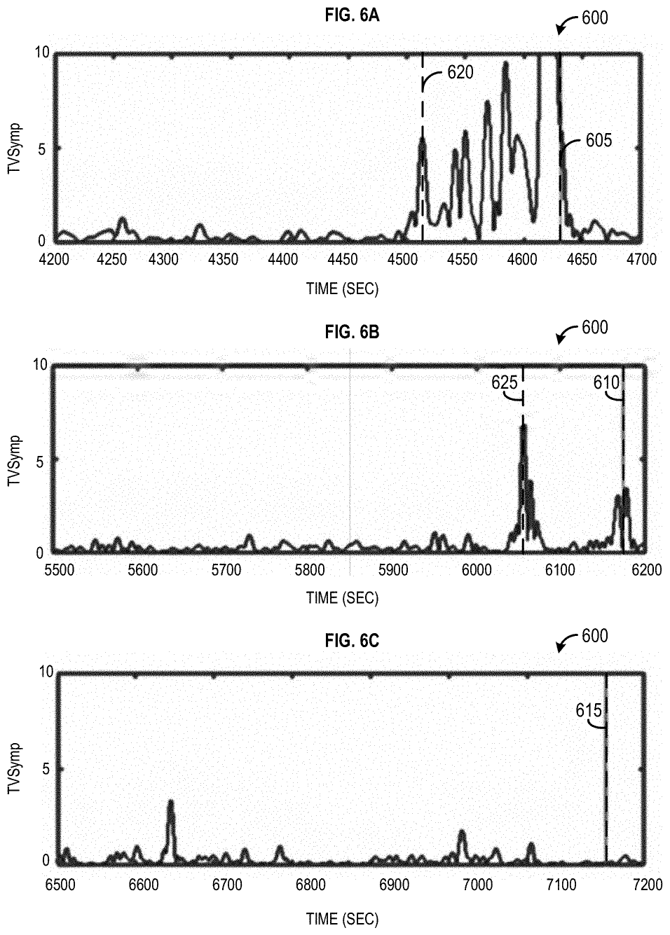

FIGS. 6A-6C show an example graphical representation 600 of TVSymp values (e.g., time-varying index values) associated with three different users. For example, FIG. 6A is representative of a user presenting with symptoms of oxygen toxicity (e.g., CNS-OT), such as diaphoresis. Vertical line 605 of FIG. 6A is representative of the point at which diaphoresis occurs, for example while conducting a dive beneath the surface of a body of water. For example, vertical line 620 of FIG. 6A is representative of the point at which the TVSymp value for the first user (e.g., time-varying index value) increases to a level above a threshold level (e.g., a static or dynamic threshold level). This increase of the TVSymp value above the threshold level may indicate that the TVSymp level for the first user satisfies the threshold and indicates oxygen toxicity is beginning to occur in the first user during the dive.

FIG. 6B is representative of a second user presenting with symptoms of oxygen toxicity (e.g., CNS-OT). Vertical line 610 of FIG. 6B is representative of the point at which diaphoresis occurs in the second user, for example, while conducting a dive beneath the surface of a body of water. For example, vertical line 625 of FIG. 6B is representative of the point at which the TVSymp value for the second user (e.g., time-varying index value) increases to a level above a threshold level (e.g., a static or dynamic threshold level) while the second user is conducting the dive. This increase of the TVSymp value above the threshold level may indicate that the TVSymp level for the second user satisfies the threshold and indicates oxygen toxicity is beginning to occur in the second user during the dive.

FIG. 6C is representative of a third user presenting without symptoms of oxygen toxicity (e.g., CNS-OT). Vertical line 615 is representative of the point at which the third user's dive (or time beneath the surface of a body of water) ends without showing any symptoms of oxygen toxicity or CNS-OT, based on the TVSymp values for the third user.

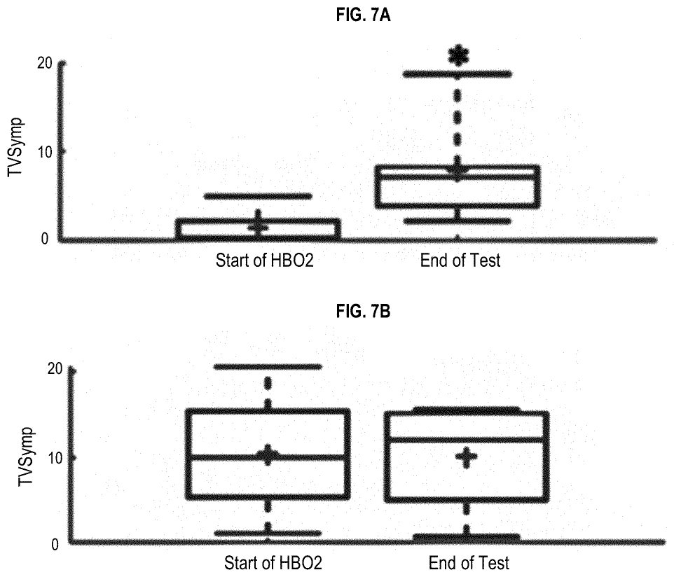

FIGS. 7A and 7B show example box plots associated with the existence of oxygen toxicity systems (e.g., CNS-OT symptoms). For example, FIG. 7A is representative of users showing symptoms of oxygen toxicity (e.g., CNS-OT). As another example, FIG. 7B is representative of users not showing symptoms of oxygen toxicity (e.g., CNS-OT). For each of FIGS. 7A and 7B it is understood that an asterisk(*) may denote a significant difference to Start of HBO2.

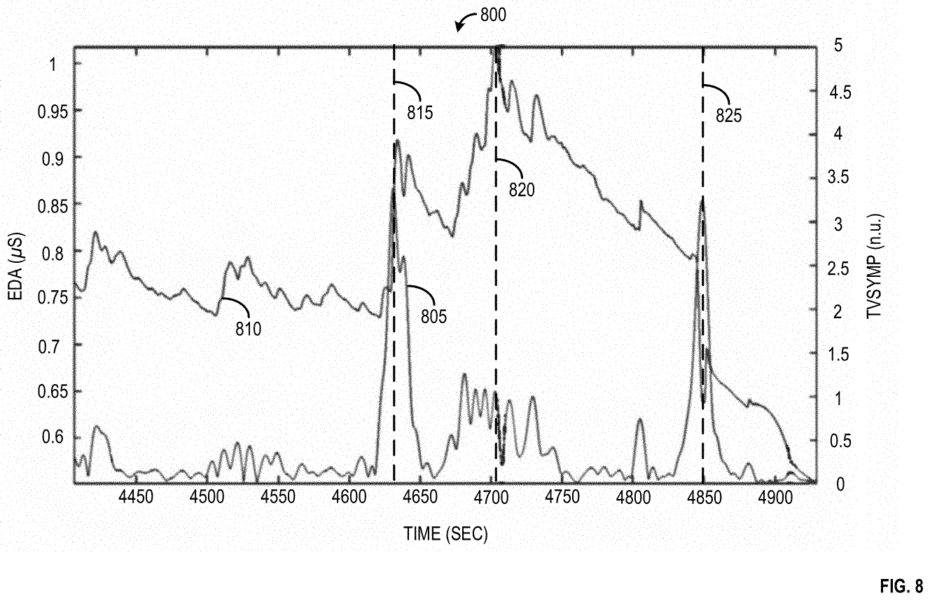

FIG. 8 shows an example graphical representation 800 of TVSymp index values 805 and values associated with raw EDA data 810. TVSymp index values of the EDA data (e.g., time-varying index values) may be obtained by determining a time-varying spectral density of the EDA data obtained from the one or more EDA sensors 120 of FIG. 1 in a frequency range associated with a sympathetic nervous system of a user (e.g., the user 101). For example, the frequency range associated with a sympathetic nervous system of a user may be 0.15-0.25 Hz. TVSymp index values may be based on determining how spectral amplitudes in the sympathetic nervous system of the user's frequency band may change with time. Based on the EDA data, and specific to FIG. 8, there may be a several-fold increase in TVSymp index values prior to when oxygen may be turned off for the user during a dive due to symptoms determined to be based on oxygen toxicity of the user. In comparison to the representative values associated with vertical line 815 and vertical line 820, an approximate seven-fold increase in TVSymp index values for the user in the span of approximately 90 seconds may be determined during the user's dive. For example, TVSymp index values at vertical line 825 shows another significant increase in TVSymp values for the user during the dive.

The TVSymp values at the vertical line 820 may also be representative of the point in time wherein the user may have developed oxygen toxicity (e.g., CNS-OT) symptoms. For example, TVSymp index values at vertical line 820 may also be representative of the point in time wherein the user was removed from oxygen (e.g., the user is no longer breathing or being provided molecular oxygen (O₂) at increased partial pressures). For example, symptoms associated with indications that the user may have developed oxygen toxicity (e.g., CNS-OT) may include one or more of hand twitching, palpitations, and/or visual disturbances.

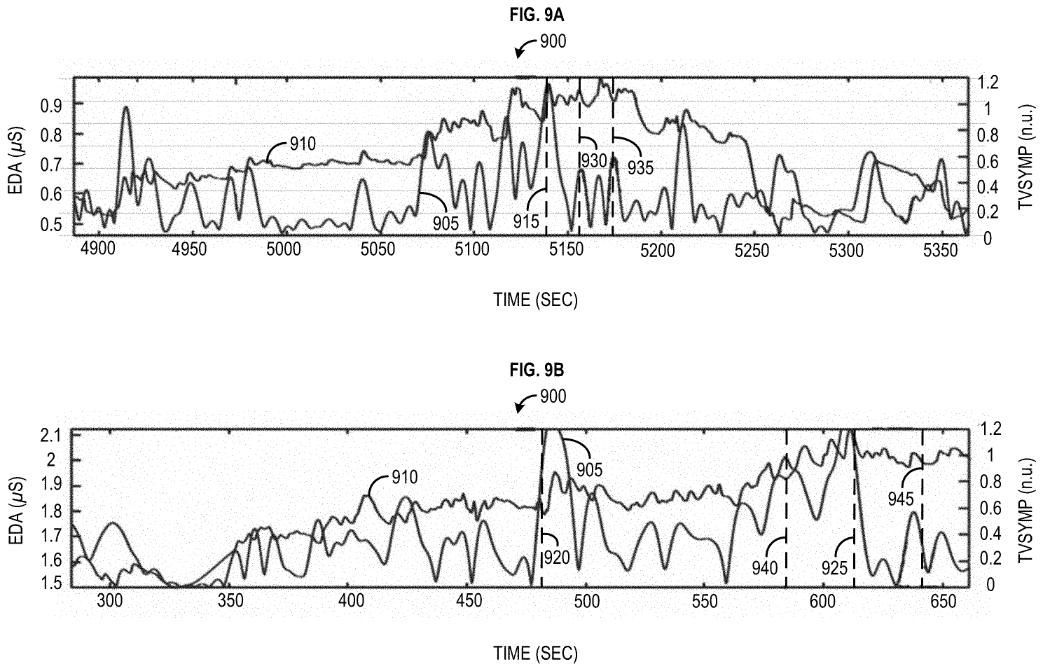

FIGS. 9A and 9B show an example graphical representation 900 of TVSymp index values 905 (e.g., time-varying index values) and values associated with raw EDA data 910. TVSymp index values may be obtained by determining a time-varying spectral density of EDA data obtained from the one or more EDA sensors 120 of FIG. 1 in a frequency range associated with a sympathetic nervous system of a user (e.g., the user 101). It is understood, however, that the graphical representation 800 of FIG. 8 depicts the results of a user that is different than the user associated with the graphical representation of FIGS. 9A and 9B. TVSymp index values at vertical lines 915-925 may each be representative of an increase in the TVSymp index values for the user. TVSymp index values at vertical line 920 may be determined prior to the user showing symptoms that indicate oxygen toxicity in the user.

Varying symptoms associated with the user may arise at different points in time after the point in time wherein the user is no longer breathing (e.g., is removed from) molecular oxygen (O₂) at increased partial pressures. For example, TVSymp index values at vertical line 915 may also be representative of the point in time wherein the user was removed from molecular oxygen (O₂) at increased partial pressures in FIG. 9A. TVSymp index values at vertical line 930 may be representative of the point in time at which the user may begin to suffer from gradual difficulty in concentration. As another example, TVSymp index values at vertical line 935 may represent the point in time at which the user may begin to suffer from sudden diaphoresis.

TVSymp index values at vertical line 940 may be representative of the point in time wherein the user was removed from molecular oxygen (O₂) at increased partial pressures in FIG. 9B. TVSymp index values at vertical line 945 may be representative of the point in time at which clammy skin may develop for the user.

While the TVSymp index values 805, 905 and values associated with EDA 810, 910 may provide a basis by which a prediction may be made with regard to oxygen toxicity for the user (e.g., the user 101), pre-existing data for the user may be helpful to serve as a foundational point by which to base the prediction (e.g., pre-existing data for the user may be used to determine the threshold that the TVSymp index values are compared to, so that a determination may be made if the user is experiencing oxygen toxicity).

For example, based on pre-existing data for the user (e.g., pre-existing EDA data and/or pre-existing TVSymp index values), a threshold value that may be determined that indicates whether oxygen toxicity is beginning to occur in the user and/or whether a seizure is likely to occur in the user. In consideration of this, the method for predicting oxygen toxicity and/or the potential for a seizure in the user may further include implementing machine learning techniques so that a classification may be made regarding whether an increase in the TVSymp index values for a user is a result of an increase in a sympathetic nervous system response to oxygen toxicity (e.g., CNS-OT) symptom(s). For example, by the implementation of machine learning techniques, the threshold value may be dynamically determined in the absence of pre-existing data (e.g., pre-existing EDA data and/or pre-existing TVSymp index values) for the user.

In addition, it may be beneficial to remove a portion of the EDA data prior to determining TVSymp index values based on the EDA data. For example, EDA data collected during certain motions of the user may cause motion artifacts within the EDA data. The EDA data with motion artifacts may distort the EDA data, and may cause inaccurate results based on inaccurately created time-varying index values. In consideration of this, machine learning and deep learning may be utilized to detect motion artifacts in the EDA data. For example, machine learning and/or deep learning models may be trained to classify clean (e.g., accurate) EDA data in comparison to corrupted EDA data that contains or was generated based on motion artifacts. The clean data may then be kept to calculate TVSymp index values while the corrupted EDA data may be discarded and not used for calculating TVSymp index values for the user.

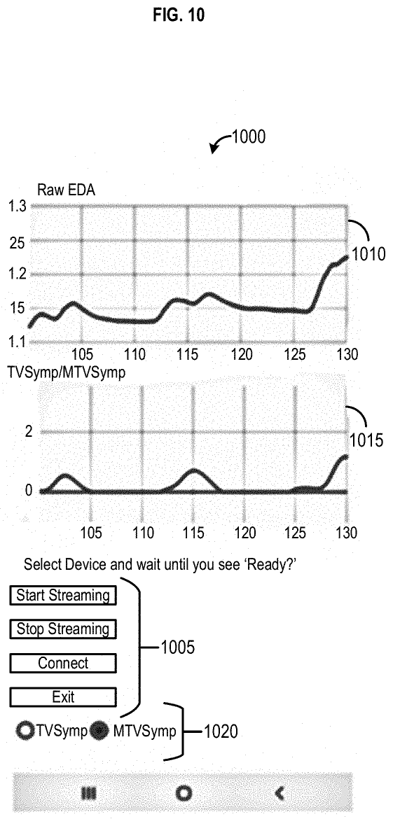

FIG. 10 shows a user interface (UI) 1000 by which a processing application may be operated by. For example, the processing application may be a part of or accessible by and used by the computing device 102, the user device 103A or any other device described herein. In addition, the UI 1000 may be displayed on a display associated with the computing device 102 (e.g., the display device 116), a display associated with the user device 103A, or a display associated with any other device described herein. For example, the processing application may process EDA data. The processing application may be configured to collect and/or process EDA data in real-time. For example, the processing application may collect and/or process EDA data by being included on and/or communicating with the user device 103A described in FIGS. 1 and 2, for example.

The UI 1000 may provide a user (e.g., the user 101) with options that the user may select in response to a prompt. For example, the prompt may indicate that the processing application is ready to process a request 1005. As an example, the request 1005 may be to start streaming EDA data, stop streaming EDA data, connect to the user device 103A, or to exit. However, additional and/or different requests may be included in the request 1005 on the UI 1000.

The processing application may receive raw EDA data. For example, the processing application may receive the raw EDA data from the EDA sensors 120 and/or the user device 103A. The UI 1000 may display the raw EDA data in a graphical format on a first portion 1010 of the UI 1000. The processing application may implement various algorithms to display either a TVSymp or MTVSymp determination based on the raw EDA data as well. For example, the UI 1000 may display the TVSymp or MTVSymp determination of the raw EDA data in a graphical format on a second portion 1015 of the UI 1000.

The UI 1000 may also provide the user with an option to select which algorithmic associated display may be displayed in the second portion 1015 of the UI 1000. For example, the UI 1000 may allow for the user to select (e.g., at a selection portion 1020 of the UI 1000) TVSymp or MTVSymp (e.g., time varying index values) as the algorithm by which to calculate, based on the raw EDA. As an example, the processing application may implement any algorithm to interpret the raw EDA.

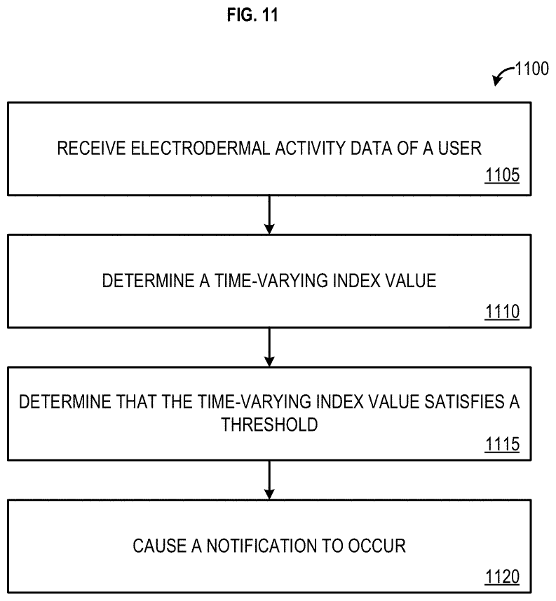

FIG. 11 shows a flowchart of an example method 1100 for the detection of seizure and/or oxygen toxicity related symptoms in a user (e.g., a diver or submariner). The method 1100 may be performed by the computing device 102 and/or any of the user devices 103A-C.

At step 1105, electrodermal activity (EDA) data of a user (e.g., the user 101) may be received. For example, the EDA data may be received while the user 101 is diving or otherwise beneath the surface of a body of water. For example, the EDA data may be received from one or more sensors, such as the EDA sensor(s) 120 of FIG. 1. For example, the EDA data may comprise data corresponding to one or more physiological signals derived from sweat gland activity of the user 101. As an example, the EDA data may be or may be associated with information that may be indicative of one or more physiological signals of the user 101. The one or more physiological signals may be derived from sweat gland activity of the user 101 and received from the EDA sensor(s) 120, or probes associated with the sensors 120, contacting the skin of the user 101.

For example, other sensor data associated with the user 101 may be received. The other sensor data may include one or more of electromyographical data readings of the user 101, electrocardiographical data readings of the user, and accelerometer data readings of the user 101. The electromyographical data readings may be received from one or more electromyography sensor(s), the electrocardiographical data readings may be received from one or more electrocardiography sensor(s), and the accelerometer data may be received from an accelerometer 122. For example, the accelerometer 122 may be a tri-axial accelerometer. For example, the accelerometer may determine movement of the user 101. For example, the movement data of the user 101 may be used to determine portions of the EDA data which may not be desirable to use due to the potential for the motion causing erroneous time-varying index values (e.g., TVSymp or MTVSymp). Each of the electromyography sensor, the electrocardiography sensor, and the accelerometer 122 may be included with the same device (e.g., the user device 103A) as the one or more EDA sensors 120 or may be part of and received from another device, such as another wearable device or another one of the user devices 103B-C. Each of the EDA data, the accelerometer data, the electromyographical data, and the electrocardiographical data may be received via a wired or wireless (e.g., Bluetooth®, BLE, RF (e.g., electromagnetic RF), WiFi, or any other known wireless communication protocol) communication. For example, EDA data and any of the other data may be sent by the user device 103A. For example, the EDA data and the other data may be sent by the user device 103A as part of an internal transmission from the respective sensors to another portion of the user device 103A. In another example, the user device 103A may send the EDA data and the other sensor data to the computing device 102 or another user device 103B-C via the wired or wireless communication.

A determination may be made as to whether all or portions of the received EDA data is valid. For example, the determination may be made by the computing device 102, the user device 103A or any other device described herein. For example, EDA data received while the user 101 is performing a certain amount of movement or exertion may not be useful in the analysis. For example, accelerometer data may be received from an accelerometer 122. For example, the accelerometer may be associated with the movement of the user 101. The accelerometer data may be compared to a motion threshold. A determination may be made as to if all or portions of the accelerometer data satisfy (e.g., is less than or less than or equal to or is greater than or greater than or equal to) the motion threshold. For example, if a first portion of the accelerometer data does not satisfy (e.g., the accelerometer data is less than or less than or equal to) the motion threshold, a determination may be made that the EDA data associated with (e.g., collected at the same time as) the first portion of the accelerometer data is valid EDA data. For example, if a second portion of the accelerometer data satisfies (e.g., the accelerometer data is greater than or greater than or equal to) the motion threshold, a determination may be made that the EDA data associated with the second portion of the accelerometer data is invalid EDA data. For example, satisfying the motion threshold may be switched from greater than or greater than or equal to, to less than or less than or equal to and the determination of which EDA data is valid and invalid may be similarly inverted (e.g., valid data satisfies the threshold and invalid data does not satisfy the threshold) in certain examples. For example, the second portion of the EDA data comprising invalid data may be removed from further analysis and the first portion of the EDA data comprising valid data may be further analyzed and processed.

At step 1110, a time-varying index value may be determined. For example, the time-varying index value may be determined by the computing device 102, the user device 103A or any other device described herein. For example, a time-varying index value may be determined based on the EDA data. For example, the time-varying index value may be determined based on the valid EDA data (e.g., the first portion of the EDA data). The time-varying index value may be determined using a frequency complex demodulation, for example. For example, the time-varying index value may be one or more of TVSymp of the EDA data or MTVSymp of the EDA data. For example, the time-varying index value may serve as a basis by which an oxygen toxicity of the user 101 is determined.

At step 1115, a determination may be made that the time-varying index value satisfies a threshold (e.g., a threshold value). For example, the determination may be made by the computing device 102, the user device 103A or any other device described herein. For example, a plurality of time-varying index values may be determined from the EDA data of the user 101. Each of the plurality of time-varying index values may be compared to the threshold value. A determination may be made, based on the comparison, whether the particular time-varying index value satisfies (e.g., is greater than or greater than or equal to) the threshold. If the time-varying index value satisfies the threshold it may indicate oxygen toxicity in the user 101. If the time varying index value does not satisfy the threshold, it may indicate no oxygen toxicity in the user 101. By evaluating time-varying index values against threshold values, oxygen toxicity in users may be determined a significant amount of time before negative physical symptoms associated with oxygen toxicity begin to occur in the user. This early indication of oxygen toxicity may allow the user to discontinue breathing, by the user, breathing molecular oxygen (O₂) (e.g., 100% O₂ air mixture) at increased partial pressures before more significant symptoms (e.g., seizures, diaphoresis, numbness in joints, etc.) associated with oxygen toxicity occur in the user.

The threshold may be static (e.g., a predetermined or preset threshold that is not adjustable) or dynamic. For example, the dynamic threshold may vary and may be determined based on one or more of historical EDA data for the user 101, historical time-varying index values for the user 101, historical EDA data for all users, or historical time-varying index values for all users. For example, the dynamic threshold may be based on the historical data of the user 101. For example, satisfying the threshold may be switched from greater than or greater than or equal to, to less than or less than or equal to and the determination of when the time-varying index value indicates oxygen toxicity in the user may be similarly inverted (e.g., not satisfying the threshold indicates oxygen toxicity in the user and satisfying the threshold indicates no oxygen toxicity in the user) in certain examples.

At step 1120, a notification is caused to be displayed. For example, the notification may be caused to be displayed by the computing device 102, the user device 103A or any other device described herein. The notification may be indicative of at least one symptom of the user, wherein the at least one symptom comprises at least one of: oxygen toxicity, a seizure, diaphoresis, numbness in joints, clammy skin and/or the like. For example, the notification that is caused to be displayed may be based on the time-varying index value satisfying the threshold (e.g., indicating oxygen toxicity in the user 101). For example, the notification may be displayed on a display of or occur at the same device that determined the time-varying index value satisfies the threshold (e.g., the computing device 102 or the user device 103A). For example, the notification, or a signal indicating the notification, may be sent to a second computing device. For example, the notification may be sent from the computing device 102 to one of the user devices 103A-C. For example, the notification may be sent from the user device 103A to the computing device 102 or to one of the other user devices 103B-C. For example, the notification may be displayed on a heads-up display (e.g., the heads-up display 104) of the second computing device. For example, the notification may be one or more of an alphanumeric notification, one or more colors of lights, a sound, or a vibration.

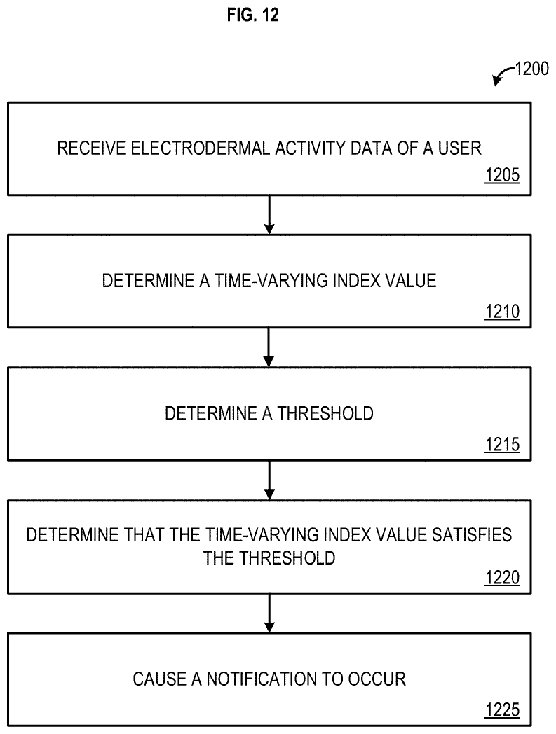

FIG. 12 shows a flowchart of an example method 1200 for the detection of seizure and/or oxygen toxicity related symptoms in a user (e.g., a diver or submariner). The method 1200 may be performed by the computing device 102 and/or any of the user devices 103A-C.

At step 1205, electrodermal activity (EDA) data of a user (e.g., the user 101) may be received. For example, the EDA data may be received while the user 101 is diving or otherwise beneath the surface of a body of water. For example, the EDA data may be received from one or more sensors, such as the EDA sensor(s) 120 of FIG. 1. For example, the EDA data may comprise data corresponding to one or more physiological signals derived from sweat gland activity of the user 101. As an example, the EDA data may be or may be associated with information that may be indicative of one or more physiological signals of the user 101. The one or more physiological signals may be derived from sweat gland activity of the user 101 and received from the EDA sensor(s) 120, or probes associated with the sensors 120, contacting the skin of the user 101.

For example, other sensor data associated with the user 101 may be received. The other sensor data may include one or more of electromyographical data readings of the user 101, electrocardiographical data readings of the user, and accelerometer data readings of the user 101. The electromyographical data readings may be received from one or more electromyography sensor(s), the electrocardiographical data readings may be received from one or more electrocardiography sensor(s), and the accelerometer data may be received from an accelerometer 122. For example, the accelerometer 122 may be a tri-axial accelerometer. For example, the accelerometer may determine movement of the user 101. For example, the movement data of the user 101 may be used to determine portions of the EDA data which may not be desirable to use due to the potential for the motion causing erroneous time-varying index values (e.g., TVSymp or MTVSymp). Each of the electromyography sensor, the electrocardiography sensor, and the accelerometer 122 may be included with the same device (e.g., the user device 103A) as the one or more EDA sensors 120 or may be part of and received from another device, such as another wearable device or another one of the user devices 103B-C. Each of the EDA data, the accelerometer data, the electromyographical data, and the electrocardiographical data may be received via a wired or wireless (e.g., Bluetooth®, BLE, RF (e.g., electromagnetic RF), WiFi, or any other known wireless communication protocol) communication. For example, EDA data and any of the other data may be sent by the user device 103A. For example, the EDA data and the other data may be sent by the user device 103A as part of an internal transmission from the respective sensors to another portion of the user device 103A. In another example, the user device 103A may send the EDA data and the other sensor data to the computing device 102 or another user device 103B-C via the wired or wireless communication.

A determination may be made as to whether all or portions of the received EDA data is valid. For example, the determination may be made by the computing device 102, the user device 103A or any other device described herein. For example, EDA data received while the user 101 is performing a certain amount of movement or exertion may not be useful in the analysis. For example, accelerometer data may be received from an accelerometer 122. For example, the accelerometer may be associated with the movement of the user 101. The accelerometer data may be compared to a motion threshold. A determination may be made as to if all or portions of the accelerometer data satisfy (e.g., is less than or less than or equal to or is greater than or greater than or equal to) the motion threshold. For example, if a first portion of the accelerometer data does not satisfy (e.g., the accelerometer data is less than or less than or equal to) the motion threshold, a determination may be made that the EDA data associated with (e.g., collected at the same time as) the first portion of the accelerometer data is valid EDA data. For example, if a second portion of the accelerometer data satisfies (e.g., the accelerometer data is greater than or greater than or equal to) the motion threshold, a determination may be made that the EDA data associated with the second portion of the accelerometer data is invalid EDA data. For example, satisfying the motion threshold may be switched from greater than or greater than or equal to, to less than or less than or equal to and the determination of which EDA data is valid and invalid may be similarly inverted (e.g., valid data satisfies the threshold and invalid data does not satisfy the threshold) in certain examples. For example, the second portion of the EDA data comprising invalid data may be removed from further analysis and the first portion of the EDA data comprising valid data may be further analyzed and processed.

At step 1210, a time-varying index value may be determined. For example, the time-varying index value may be determined by the computing device 102, the user device 103A or any other device described herein. For example, a time-varying index value may be determined based on the EDA data. For example, the time-varying index value may be determined based on the valid EDA data (e.g., the first portion of the EDA data). The time-varying index value may be determined using a frequency complex demodulation, for example. For example, the time-varying index value may be one or more of TVSymp of the EDA data or MTVSymp of the EDA data. For example, the time-varying index value may serve as a basis by which an oxygen toxicity of the user 101 is determined.

At step 1215, a threshold may be determined. For example, the threshold may be determined based on historical data associated with the user. For example, the threshold may be determined based on one or more of historical EDA data for the user or historical time-varying index values for the user. As another example, the threshold may be determined via a machine learning module. The machine learning module may be a component of the computing device 102 or any of the user devices 103A-C, for example. For example, the machine learning module may use machine learning techniques to determine the threshold based on dive habits of the user. The dive habits of the user may be indicative of the user's typical motion and oxygen consumption, for example. For example, the dive habits of the user may include one or more of historical EDA data for the user, historical time-varying index values for the user, or historical accelerometer data for the user. The implementation of machine learning in the determination of the threshold may include techniques so that a classification may be made regarding whether an increase in the TVSymp index values for the user is a result of an increase in a sympathetic nervous system response to oxygen toxicity (e.g., CNS-OT) symptom(s). For example, the threshold may be determined in the absence of pre-existing data for the user. For example, the implementation of machine learning in the determination of the threshold may also include techniques so that a classification may be made regarding whether an increase in the time-varying index values (e.g., TVSymp, MTVSymp) for the user may be indicative of oxygen toxicity and/or an impending seizure that the user may endure.

Machine learning may also be utilized to detect motion artifacts in the EDA data. For example, machine learning and/or deep learning models may be trained to classify clean (e.g., valid) EDA data in comparison to invalid EDA data that contains or was generated during motion by the user. The valid EDA data may then be used to calculate the time-varying index values (e.g., TVSymp, MTVSymp) while the invalid EDA data may be discarded and not used for calculating the time-varying index values for the user.

As another example, a motion threshold may dynamically change based on dive patterns of the user. For example, the motion threshold may dynamically change by the use of machine learning techniques based on historical acceleration data of the user.

At step 1220, a determination may be made that the time-varying index value satisfies a threshold (e.g., a threshold value). For example, the determination may be made by the computing device 102, the user device 103A or any other device described herein. For example, a plurality of time-varying index values may be determined from the EDA data of the user 101. Each of the plurality of time-varying index values may be compared to the threshold value. A determination may be made, based on the comparison, whether the particular time-varying index value satisfies (e.g., is greater than or greater than or equal to) the threshold. If the time-varying index value satisfies the threshold it may indicate oxygen toxicity in the user 101. If the time varying index value does not satisfy the threshold, it may indicate no oxygen toxicity in the user 101. By evaluating time-varying index values against threshold values, oxygen toxicity in users may be determined by a significant amount of time before negative physical symptoms associated with oxygen toxicity begin to occur in the user. This early indication of oxygen toxicity may allow the user to discontinue breathing, by the user, breathing molecular oxygen (O₂) (e.g., 100% O₂ air mixture) at increased partial pressures before more significant symptoms (e.g., seizures, diaphoresis, numbness in joints, etc.) associated with oxygen toxicity occur in the user.

The threshold may be static (e.g., a predetermined or preset threshold that is not adjustable) or dynamic. For example, the dynamic threshold may vary and may be determined based on one or more of historical EDA data for the user 101, historical time-varying index values for the user 101, historical EDA data for all users, or historical time-varying index values for all users. For example, the dynamic threshold may be based on the historical data of the user 101. For example, satisfying the threshold may be switched from greater than or greater than or equal to, to less than or less than or equal to and the determination of when the time-varying index value indicates oxygen toxicity in the user may be similarly inverted (e.g., not satisfying the threshold indicates oxygen toxicity in the user and satisfying the threshold indicates no oxygen toxicity in the user) in certain examples.

At step 1225, a notification is caused to be displayed. For example, the notification may be caused to be displayed by the computing device 102, the user device 103A or any other device described herein. The notification may be indicative of at least one symptom of the user, wherein the at least one symptom comprises at least one of: oxygen toxicity, a seizure, diaphoresis, numbness in joints, clammy skin, and/or the like. For example, the notification that is caused to be displayed may be based on the time-varying index value satisfying the threshold (e.g., indicating oxygen toxicity in the user 101). For example, the notification may be displayed on a display of or occur at the same device that determined the time-varying index value satisfies the threshold (e.g., the computing device 102 or the user device 103A). For example, the notification, or a signal indicating the notification, may be sent to a second computing device. For example, the notification may be sent from the computing device 102 to one of the user devices 103A-C. For example, the notification may be sent from the user device 103A to the computing device 102 or to one of the other user devices 103B-C. For example, the notification may be displayed on a heads-up display (e.g., the heads-up display 104) of the second computing device. For example, the notification may be one or more of an alphanumeric notification, one or more colors of lights, a sound, or a vibration.

While the methods and systems have been described in connection with preferred embodiments and specific examples, it is not intended that the scope be limited to the particular embodiments set forth, as the embodiments herein are intended in all respects to be illustrative rather than restrictive.

While specific configurations have been described, it is not intended that the scope be limited to the particular configurations set forth, as the configurations herein are intended in all respects to be possible configurations rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered with only a true scope and spirit being indicated by the following claims.

What is claimed is:

1. A method comprising:
receiving, by a computing device from a sensor, electrodermal activity data of a user;
determining, based on the electrodermal activity data, a time-varying index value;
determining that the time-varying index value satisfies a threshold or a machine learning classification; and
causing, based on the time-varying index value satisfying the threshold or the machine learning classification, a notification to occur.

2. The method of claim 1, further comprising determining, based on the time-varying index value satisfying the threshold or the machine learning classification, an oxygen toxicity in the user.

3. The method of claim 1, wherein the electrodermal activity data comprises one or more physiological signals derived from sweat gland activity of the user.

4. The method of claim 1, wherein causing the notification to occur comprises sending, to a second computing device, the notification to be displayed on a heads-up display of the second computing device.

5. The method of claim 1, further comprising:
determining a first portion of the electrodermal activity data comprises valid data and a second portion of the electrodermal activity data comprises invalid data,
wherein determining, based on the electrodermal activity data, the time-varying index value comprises determining, based on the first portion of the electrodermal activity data, the time-varying index value.

6. The method of claim 5, further comprising:
receiving, by the computing device, accelerometer data for the user; and
determining, at least a portion of the accelerometer data satisfies a motion threshold, wherein the at least the portion of the accelerometer data corresponds to at least a portion of the second portion of the electrodermal activity data,
wherein determining the second portion of the electrodermal activity data comprises invalid data comprises determining, based on the at least the portion of the accelerometer data satisfying the motion threshold, the second portion of the electrodermal activity data comprises invalid data.

7. The method of claim 1, wherein the time-varying index value is determined using a frequency complex demodulation.

8. The method of claim 1, wherein the threshold is a dynamic threshold and wherein the dynamic threshold is determined based on historical data of the user or via the machine learning classification

9. The method of claim 1, wherein the notification is indicative of at least one symptom of the user, wherein the at least one symptom comprises at least one of: oxygen toxicity, a seizure, diaphoresis, numbness in joints, or clammy skin.

10. A method comprising:
receiving, by a computing device from a sensor, electrodermal activity data of a user;
determining, based on the electrodermal activity data, a time-varying index value;
determining, via a machine learning module and based on historical data of the user, a threshold;
determining that the time-varying index value satisfies the threshold or a machine learning classification; and
causing, based on the time-varying index value satisfying the threshold or the machine learning classification, a notification to occur.

11. The method of claim 10, further comprising training, based on the historical data of the user, the machine learning module to determine that oxygen toxicity is occurring in the user.

12. The method of claim 10, further comprising determining, based on the time-varying index value satisfying the threshold or the machine learning classification, an oxygen toxicity in the user.

13. The method of claim 10, wherein the electrodermal activity data comprises one or more physiological signals derived from sweat gland activity of the user.

14. The method of claim 10, wherein causing the notification to occur comprises sending, to a second computing device, the notification to be displayed on a heads-up display of the second computing device.

15. The method of claim 10, further comprising:
determining a first portion of the electrodermal activity data comprises valid data and a second portion of the electrodermal activity data comprises invalid data,
wherein determining, based on the electrodermal activity data, the time-varying index value comprises determining, based on the first portion of the electrodermal activity data, the time-varying index value.

16. The method of claim 15, further comprising:
receiving, by the computing device, accelerometer data for the user; and
determining, at least a portion of the accelerometer data satisfies a motion threshold, wherein the at least the portion of the accelerometer data corresponds to at least a portion of the second portion of the electrodermal activity data,
wherein determining the second portion of the electrodermal activity data comprises invalid data comprises determining, based on the at least the portion of the accelerometer data satisfying the motion threshold, the second portion of the electrodermal activity data comprises invalid data.

17. The method of claim 10, further comprising training, based on the historical data of the user, the machine learning module to determine that a seizure associated with the user is likely to occur.

18. The method of claim 10, wherein the notification is indicative of at least one symptom of the user, wherein the at least one symptom comprises at least one of: oxygen toxicity, a seizure, diaphoresis, numbness in joints, or clammy skin.

19. The method of claim 10, wherein the time-varying index value is determined using a frequency complex demodulation.

20. An apparatus comprising:
one or more processors; and
a memory storing processor-executable instructions that, when executed by the one or more processors, cause the apparatus to:
receive, from a sensor, electrodermal activity data of a user;
determine, based on the electrodermal activity data, a time-varying index value;
determine that the time-varying index value satisfies a threshold or a machine learning classification; and
cause, based on the time-varying index value satisfying the threshold or the machine learning classification, a notification to occur.
